(19) United States
(12) Patent Application Publication
(10) Pub. No.: US 2010/0168741 A1
(43) Pub. Date: Jul. 1, 2010

(54) SURGICAL OPERATION APPARATUS

(76) Inventors: Hideo Sanai, Hachioji-shi (JP); Norikiyo Shibata, Yamato-shi (JP); Satoshi Honma, Hino-shi (JP); Yusuke Tadami, Hachioji-shi (JP)

Correspondence Address: OSTROLENK FABER GERB & SOFFEN, 1180 AVENUE OF THE AMERICAS, NEW YORK, NY, 100368403, US

(21) Appl. No.: 12/344,769

(22) Filed: Dec. 29, 2008

(51) Int. Cl. A61B 18/00; A61B 18/14

(52) U.S. Cl. 606/42; 606/169

(57) ABSTRACT

A surgical operation apparatus includes a probe to which ultrasonic vibration is transmitted, and a flat plate-like blade which is formed at a distal end part of the probe, and can simultaneously output ultrasonic vibration and a high-frequency current, wherein the blade includes a contact area reduction part for reducing an area of contact with the living tissue to thereby increase a current density of the high-frequency current.

### BACKGROUND OF THE INVENTION

The present invention relates to a surgical operation apparatus for performing surgical procedures such as coagulation/incision of living tissue by utilizing ultrasonic energy and high-frequency energy.

As an example of a general ultrasonic treating apparatus for performing treatments such as coagulation/incision and the like of living tissue by utilizing an ultrasonic wave, there is, for example, a surgical operation apparatus disclosed in Jpn. Pat. Appln. KOKAI Publication No. 2000-308644 (Pat. Document 1). This apparatus is provided with an end effector for transmitting ultrasonic energy and high-frequency energy at a distal end part of a waveguide of an acoustic assembly body. This is a device for emulsifying and cauterizing living tissue by simultaneously supplying ultrasonic energy and high-frequency energy to the end effector, and bringing the end effector into contact with the living tissue.

Further, in Jpn. Pat. Appln. KOKAI Publication No. 2007-21196 (Pat. Document 2), an electrosurgical electrode in which a blade of an electrosurgical scalpel is provided with a conical protrusion or a concavity at a distal end thereof is disclosed. Here, a configuration is shown in which an outer surface of an electrosurgical electrode is coated with a silver alloy, whereby heat generation at the incised tissue surface, and denaturation of the incised tissue surface caused by the electrosurgical electrode when the electrosurgical electrode is brought into contact with the living tissue is minimized, and damage to the living tissue to be incised is reduced.

Further, in Jpn. Pat. Appln. KOKAI Publication No. 2005-278759 (Pat. Document 3), a high-frequency surgical instrument for staunching blood by supplying a high-frequency current to a high-frequency electrode in a state where the high-frequency electrode is kept in contact with living tissue, and cauterizing/coagulating the living tissue is shown. Here, a configuration is shown in which a through-hole having a lightning hole-like shape is formed in a high-frequency electrode having a planar shape, whereby it is possible to perform cauterization/coagulation smoothly while exerting sufficient cauterizing capability and coagulating capability, and securing a current density at the contact surface of the high-frequency electrode.

Further, in Jpn. Pat. Appln. KOKAI Publication No. 2005-329095 (Pat. Document 4), a high-frequency surgical instrument for endoscope is shown. Here, a configuration is shown in which a high-frequency electrode is formed into a spatula-like shape, and an uneven part is provided on a surface on one side thereof as a nonslip surface. This is a surgical instrument enabling mucous membrane incision and mucous membrane detachment by one instrument by means of the spatula-like high-frequency electrode.

Devices that enable coagulation/incision of viscera/tissue with less bleeding by simultaneously outputting ultrasonic energy and high-frequency energy are developed as surgical instruments. These devices enable even coagulation/incision of a parenchymatous viscus (such as the liver) that has been impossible by the use of the conventional electrosurgical scalpel. When a parenchymatous viscus is incised, the incision is performed in a state where a distal end of the surgical instrument is inserted into the living tissue. In general, a side surface of an operating part A1 of a distal end of a probe has a flat shape as shown in FIG. 33. Accordingly, when the distal end of the operating part Al of the probe is inserted into the living tissue H, a procedure such as coagulation/incision or the like is performed in a state where an area of contact between the operating part Al of the probe and the living tissue H is large.

### BRIEF SUMMARY OF THE INVENTION

A surgical operation apparatus according to an aspect of the present invention comprises: a probe to which ultrasonic vibration is transmitted; and a flat plate-like blade which is formed at a distal end part of the probe, and can simultaneously output ultrasonic vibration and a high-frequency current, wherein the blade includes a contact area reduction part for reducing an area of contact with the living tissue to thereby increase a current density of a high-frequency current.

Preferably, the contact area reduction part includes, on flat surfaces on both sides of the blade, convex parts outwardly protruded from positions on each of the flat surfaces, and each of the convex parts is provided to extend in at least one of a direction perpendicular to an axial direction of the blade, and a direction obliquely intersecting the axial direction of the blade.

Preferably, as the convex parts, a plurality of linear protruding parts extended in a direction perpendicular to the axial direction of the blade are arranged in line in the axial direction of the blade on the flat surfaces on both sides of the blade.

Preferably, as the convex parts, a plurality of linear protruding parts extended in an oblique direction obliquely inclined with respect to a direction perpendicular to the axial direction of the blade are arranged in line in the axial direction of the blade on the flat surfaces on both sides of the blade.

Preferably, as the convex parts, a plurality of hemispherical protruding parts are saliently provided on the flat surfaces on both sides of the blade.

Preferably, the contact area reduction part includes inwardly depressed concave parts on the flat surfaces on both sides of the blade.

Preferably, the concave part includes a flat surface in at least one of a direction perpendicular to the axial direction of the blade, and a direction obliquely intersecting the axial direction of the blade.

Preferably, the concave part is able to produce cavitation when the ultrasonic vibration is output.

Preferably, the contact area reduction part includes tooth parts each of which has a sawtooth shape and which are formed by continuously providing a plurality of convex parts and concave parts alternately on the outside of both edge surfaces of the blade.

Preferably, the contact area reduction part includes a hole part penetrating the blade from one of flat surfaces on both sides of the blade to the other.

Preferably, the hole part is arranged on a center axis of the blade.

Preferably, a plurality of the hole parts are provided along the center axis of the blade.

Preferably, the hole part has an oval shape or an elliptical shape elongated in the center axis direction of the blade.

Preferably, the hole part has a rhombic shape a major axis of which is arranged in a direction perpendicular to the center axis direction of the blade.

Preferably, in the probe, an antinodal position of the ultrasonic vibration is set at a distal end of the blade, and the contact area reduction part is formed within a range of a quarter-wave length of the ultrasonic vibration from the distal end position of the blade.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a side view showing the overall schematic configuration of a surgical instrument of a first embodiment of the present invention.

FIG. 2 is a longitudinal cross-sectional view showing a handpiece of the first embodiment.

FIG. 3 is a longitudinal cross-sectional view showing an operating part unit of the first embodiment.

FIG. 4 is a plan view showing a probe of the surgical instrument of the first embodiment.

FIG. 5 is a plan view showing a part D1 of the probe of FIG. 4 in an enlarging manner.

FIG. 6 is a side view of the probe of FIG. 5.

FIG. 7 is a front view showing a state of the probe of FIG. 6 viewed from the front.

FIG. 8 is a longitudinal cross-sectional view showing a usage state of the surgical instrument of the first embodiment.

FIG. 9 is a plan view showing a modification example of the probe of the surgical instrument of the first embodiment.

FIG. 10 is a side view of the probe of FIG. 9.

FIG. 11 is a plan view of a probe of a surgical instrument of a second embodiment of the present invention.

FIG. 12 is a side view of the probe of FIG. 11.

FIG. 13 is a longitudinal cross-sectional view showing a usage state of the surgical instrument of the second embodiment.

FIG. 14 is a plan view showing a probe of a surgical instrument of a third embodiment of the present invention.

FIG. 15 is a plan view showing a part D2 of the probe of FIG. 14 in an enlarging manner.

FIG. 16 is a side view of the probe of FIG. 15.

FIG. 17 is a front view showing a state of the probe of FIG. 16 viewed from the front.

FIG. 18 is a plan view showing a probe of a surgical instrument of a fourth embodiment of the present invention.

FIG. 19 is a plan view showing a part D3 of the probe of FIG. 18 in an enlarging manner.

FIG. 20 is a side view of the probe of FIG. 19.

FIG. 21 is a front view showing a state of the probe of FIG. 20 viewed from the front.

FIG. 22 is a longitudinal cross-sectional view showing a usage state of the surgical instrument of the fourth embodiment.

FIG. 23 is an explanatory view for explaining an occurrence state of cavitation occurring at a concave part of the probe of the fourth embodiment.

FIG. 24 is a perspective view showing a modification example of the probe of the surgical instrument of the fourth embodiment.

FIG. 25 is a cross-sectional view taken along line 25-25 of FIG. 24.

FIG. 26 is a plan view showing a probe of a surgical instrument of a fifth embodiment of the present invention.

FIG. 27 is a plan view showing a part D4 of the probe of FIG. 26 in an enlarging manner.

FIG. 28 is a side view of the probe of FIG. 27.

FIG. 29 is a front view showing a state of the probe of FIG. 28 viewed from the front.

FIG. 30 is a perspective view showing hole parts of the probe of the fifth embodiment.

FIG. 31 is a perspective view showing a modification example of the probe of the fifth embodiment.

FIG. 32 is an explanatory view for explaining an occurrence state of cavitation of a probe of a surgical instrument of a sixth embodiment of the present invention.

FIG. 33 is a longitudinal cross-sectional view showing a usage state of a conventional surgical instrument.

### DETAILED DESCRIPTION OF THE INVENTION

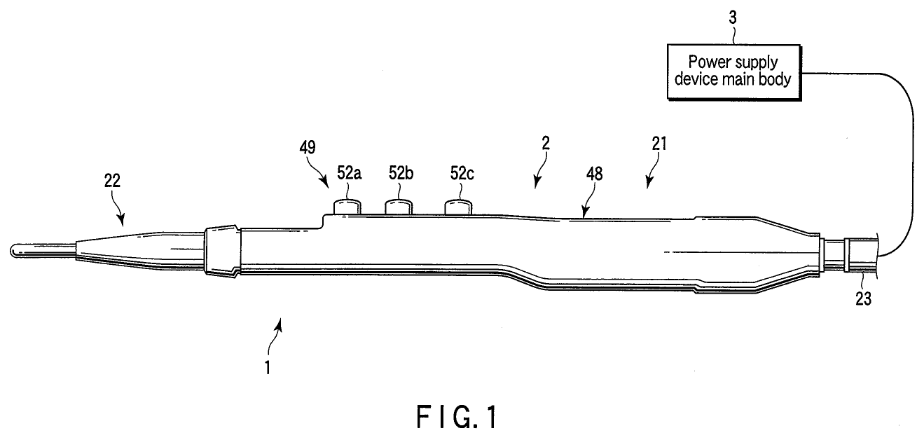

A first embodiment of the present invention will be described below with reference to FIGS. 1 to 8. FIG. 1 shows the overall schematic configuration of a surgical operation apparatus 1 of this embodiment. The surgical operation apparatus 1 of this embodiment includes a surgical instrument 2 which is a high-frequency surgical instrument of an ultrasonic-output parallel use type.

Referring to FIG. 1, the surgical instrument 2 has, as a whole, a thin and long shape, and extends in the axial direction. The surgical instrument 2 includes a handpiece 21 to be held and operated by the surgeon. An operating part unit 22 for operating on living tissue is detachably coupled to a distal end part of the handpiece 21. An end of an electric cable 23 is connected to a proximal end part of the handpiece 21. The other end of the electric cable 23 is connected to a power supply device main body 3 for driving the surgical instrument 2.

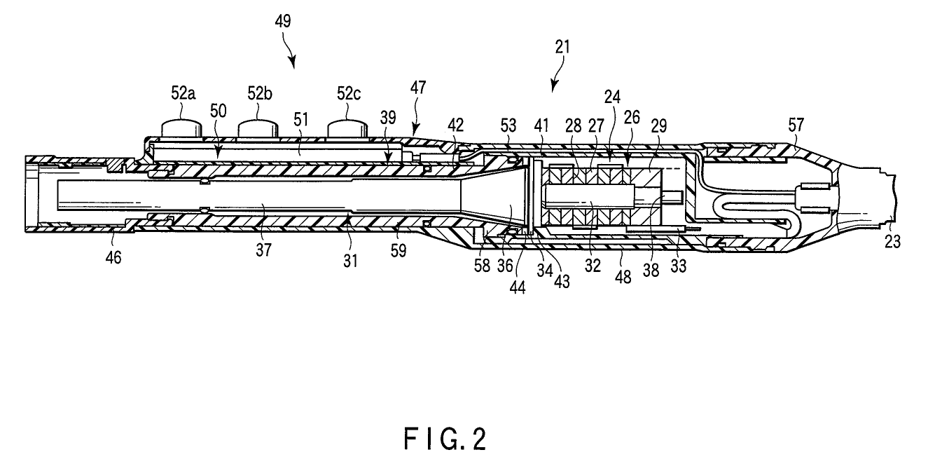

Referring to FIG. 2, a vibrator 24 is incorporated in the handpiece 21. A piezoelectric element part 26 is arranged at a proximal end part of the vibrator 24. In the piezoelectric element part 26, a plurality of piezoelectric elements 27 each having an annular plate-like shape, and a plurality of electrodes 28 are alternately superposed upon each other in the axial direction. A cylindrical backing plate 29 is superposed on the proximal end of the piezoelectric element part 26 in the axial direction. Outer diameters of the piezoelectric element 27, electrode 28, and backing plate 29 are identical with each other, and hence the piezoelectric element part 26 has a constant outer diameter D over the whole span thereof in the axial direction. A distal end face of the piezoelectric element part 26 is opposed to a proximal end face of a horn 31.

A bolt 32 is saliently provided on the proximal end face of the horn 31 to be directed to the proximal end of the handpiece in the axial direction. The bolt 32 penetrates the piezoelectric elements 27 and the electrodes 28. The backing plate 29 is screwed onto the distal end part of the bolt 32. By screwing the backing plate 29 onto the bolt 32, the piezoelectric elements 27 and the electrodes 28 are tightly held between the proximal end face of the horn 31 and the backing plate 29. To positive electrodes and negative electrodes of the plurality of electrodes 28, a distal end part of an ultrasonic cable 33 for positive electrode, and a distal end part of the ultrasonic cable 33 for negative electrode are connected, respectively. The ultrasonic cable 33 is guided to the electric cable 23, and is connected to the cable 23. A drive current is supplied to the piezoelectric element part 26 from the device main body 3 through the ultrasonic cable 33, whereby electric vibration is converted into mechanical vibration in the piezoelectric element part 26, and ultrasonic vibration is produced.

The horn 31 as a vibration transmission part has a cylindrical shape, and extends in the axial direction. A flange part 34 for fixing the horn 31 is formed at the proximal end part of the horn 31.

A distal end part of a high-frequency cable 38 is connected to the negative electrodes of the plurality of electrodes 28 of the piezoelectric element part 26. The high-frequency cable 38 is guided to the electric cable 23, and is connected to the cable 23. A high-frequency current is supplied to the piezoelectric element part 26 from the device main body 3 through the high-frequency cable 38, and a high-frequency current is made to flow through the vibrator 24.

The vibrator 24 is contained in a cylindrical inner side housing 39. The inner side housing 39 extends in the axial direction to be coaxial with the vibrator 24. Further, the inner side housing 39 is constituted of a proximal end side inner cylinder 41 and a distal end side inner cylinder 42.

The piezoelectric element part 26 is contained in the proximal end side inner cylinder 41. The ultrasonic cable 33 extended from the piezoelectric element part 26 is inserted in a through-hole formed in the inner side housing 39, and is further extended from the inner side housing 39 toward the proximal end side. The same is true of the high-frequency cable 38. On an inner circumferential surface of the proximal end side inner cylinder 41 on the distal end side, a protruding part 43 for fixation is provided in the circumferential direction. Further, a proximal end part of the distal end side inner cylinder 42 is inserted in a distal end part of the proximal end side inner cylinder 41, and is screwed into the distal end part. The distal end side inner cylinder 42 is screwed into the proximal end side inner cylinder 41, whereby the flange part 34 of the vibrator 24 is held and fixed by the protruding part 43 of the proximal end side inner cylinder 41 and a proximal end face of the distal end side inner cylinder 42. It should be noted that a spacer 44 for adjusting the position of the vibrator 24 in the axial direction is provided between a distal end face of the flange part 34 and the proximal end face of the distal end side inner cylinder 42. In this way, the vibrator 24 is fixed in the inner side housing 39 at the flange part 34 which is the nodal position of the ultrasonic vibration.

The horn 31 is contained in the distal end side inner cylinder 42. An inner diameter of the distal end side inner cylinder 42 is made slightly larger than an outer diameter of the horn 31. In the distal end side inner cylinder 42, a large diameter part 58 on the proximal end side containing therein a tapered part 36 of the horn 31, and a small diameter part 59 on the distal end side containing therein an extension part 37 of the horn 31 are formed. It should be noted that a proximal end part of a cylindrical coupling cylinder 46 is coaxially coupled to a distal end part of the distal end side inner cylinder 42.

The inner side housing 39 is contained in the outer side housing 47. The outer side housing 47 extends in the axial direction to be coaxial with the inner side housing 39. The proximal end side part of the outer side housing 47 constitutes a holding part 48 to be held by the surgeon. On the other hand, a handswitch part 49 serving as an operation part for operating the surgical instrument 2 is provided on the distal end side of the outer housing 47. The handswitch part 49 is electrically connected to the electric cable 23, and transmits signals to the device main body 3 through the electric cable 23.

In the inner side housing 39 contained in the outer side housing 47, an outer diameter of the small diameter part 59 of the distal end side inner cylinder 42 is made smaller than an outer diameter of the proximal end side inner cylinder 41, and an outer diameter of the large diameter part 58 of the distal end side inner cylinder 42. As a result of this, in the outer side housing 47, a containing space 50 is formed on the outside of the small diameter part 59 in the radial direction. In the containing space 50, a switch main body 51 of the handswitch part 49 is contained.

In the handpiece 21, an outer diameter of the handswitch part 49 is substantially identical with an outer diameter of the holding part 48, and hence the outer diameter of the handpiece 21 is substantially constant over the whole length thereof in the axial direction. Three handswitches 52a, 52b, and 52c are so provided at an outside part of the switch main body 51 in the radial direction as to allow them to protrude outwardly in the radial direction from the switch main body 51, and to be freely retractable. The handswitches 52a, 52b, and 52c are provided to protrude from the outer side housing 47. In this embodiment, in the handswitch part 49, the three handswitches 52a, 52b, and 52c are arranged in line in the axial direction from the distal end side toward the proximal end side. A switch cable 53 is extended from a proximal end part of the switch main body 51. The switch cable 53 is inserted between the outer side housing 47 and the inner side housing 39, and extended toward the proximal end side.

In the surgical instrument 2 of this embodiment, three modes are employed. By performing an operation to depress one of the three handswitches 52a, 52b, and 52c, it is possible to operate the surgical instrument in one of the three modes. The modes to be employed, and allocation of the handswitches 52a, 52b, and 52c to the modes can be arbitrarily set. For example, to the distal handswitch/intermediate handswitch/proximal handswitch 52a, 52b, and 52c, a high-frequency incision mode/high-frequency coagulation mode/coagulation-incision mode in which a high-frequency current and ultrasonic wave are simultaneously output, are respectively allocated.

To a proximal end part of the outer side housing 47, a distal end part of a proximal end housing 57 is coaxially coupled. To a proximal end part of the proximal end housing 57, a distal end part of the electric cable 23 is connected. The ultrasonic cable 33, high-frequency cable 38, and switch cable 53 extended from the proximal end part of the inner side housing 39 are introduced into the proximal end housing 57, and are then guided to the electric cable 23.

It should be noted that I the handpiece 21, sealing members such as O-rings or the like are appropriately arranged between the members to maintain the inside fluid-tight, and protect the electric elements, and hence the handpiece 21 is made compatible with autoclave sterilization using high-temperature/high-pressure steam.

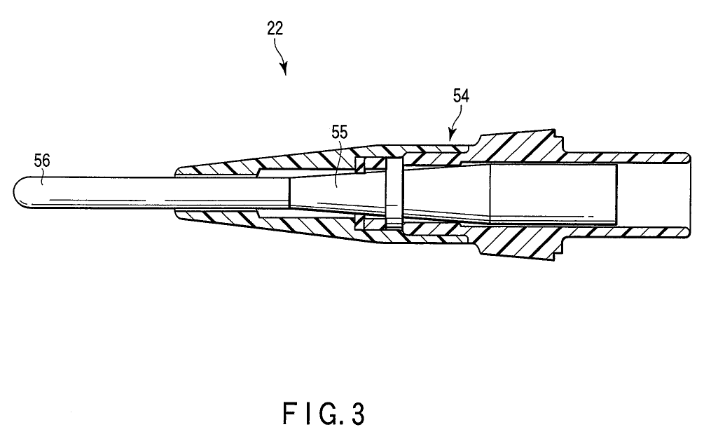

Referring to FIG. 3, the operating part unit 22 to be attached to or detached from the handpiece 21 includes a cylindrical sheath 54. A columnar probe 55 is inserted in the sheath 54, and the probe 55 is held by the sheath 54. A distal end part of the probe 55 protrudes from a distal end part of the sheath, and constitutes an operating part 56 for operating on living tissue.

A coupling mechanism for detachably and coaxially coupling the operating part unit 22 to the handpiece 21 is formed in the coupling cylinder 46 of the handpiece 21, and at a proximal end part of the sheath 54 of the operating part unit 22. When the operating part unit 22 is coupled to the handpiece 21, a proximal end part of the probe 55 of the operating part unit 22 is pressed by a distal end part of the horn 31 of the handpiece 21. The vibrator 24 of the handpiece 21 and the probe 55 of the operating part unit 22 are vibrated by ultrasonic vibration as one body. At this time, the proximal end and the distal end of the probe 55 become the antinodal positions of the vibration, and the length (L1) of the probe 55 in the axial direction becomes a half-wave length of the ultrasonic vibration. Further, by supplying a high-frequency current to the vibrator 24 of the handpiece 21, the high-frequency current is supplied to the probe 55.

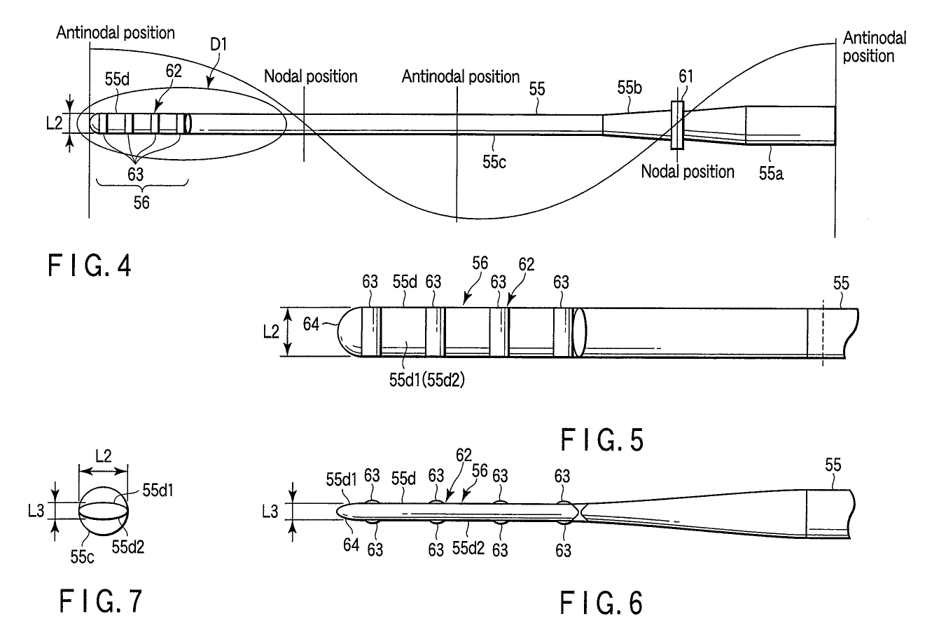

FIG. 4 shows the overall configuration of the probe 55. A large diameter part 55a having the largest diameter is provided at a proximal end part of the probe 55. At a distal end of the large diameter part 55a, a probe main body 55c having a smaller diameter than the large diameter part 55a, and having a round bar-shape is provided through a tapered part 55b having a tapering shape. Furthermore, a flange part 61 is formed at a substantially intermediate part of the tapered part 55b in the axial direction. A blade 55d having a flat plate-like shape is provided at a distal end part of the probe main body 55c. The operating part 56 described previously for operating on the living tissue is constituted of the part of this blade 55d.

The blade 55d includes a contact area reduction part 62 for reducing the area of contact with the living tissue to thereby increase the current density of the high-frequency current. As shown in FIGS. 5 and 6, the contact area reduction part 62 includes, on flat surfaces 55d1 and 55d2 on both sides of the blade 55d, a plurality of convex parts 63 outwardly protruded from positions on the flat surfaces 55d1 and 55d2. Each of the convex parts 63 is provided to extend in a direction perpendicular to the axial direction of the blade 55d.

The distal end of the blade 55d is set at an antinodal position of the ultrasonic vibration, and the rear end of the blade 55d is set at a nodal position of the ultrasonic vibration. Further, the convex parts 63 are arranged in the vicinity of the antinodal position of the ultrasonic vibration, e.g., within a range of a quarter-wave length of the ultrasonic vibration from the distal end position of the blade 55d.

Further, in this embodiment, as shown in FIG. 7, the blade 55d is formed in such a manner that a cross-sectional shape thereof is substantially elliptical. The major axis (=L2) of the ellipse is set at 3 mm, and the minor axis (=L3) thereof is set at 1 mm.

Further, a value (L3)/(L2) is set at 0.2 to 0.4 ((L3)/(L2)=0.2 to 0.4). Furthermore, at the distal end of the blade 55d, a distal end operating part 64 which is smoothly chamfered is formed. As a result of this, the blade 55d is capable of normal ultrasonic vibration, and achieves an improvement in incising capability by sharpening the operating part 56.

Furthermore, the plurality of convex parts 63 are each arranged on the flat surfaces 55d1 and 55d2 on both sides of the blade 55d at positions symmetrical with respect to the axis of the probe 55. As a result of this, transverse vibration of ultrasonic vibration can be prevented.

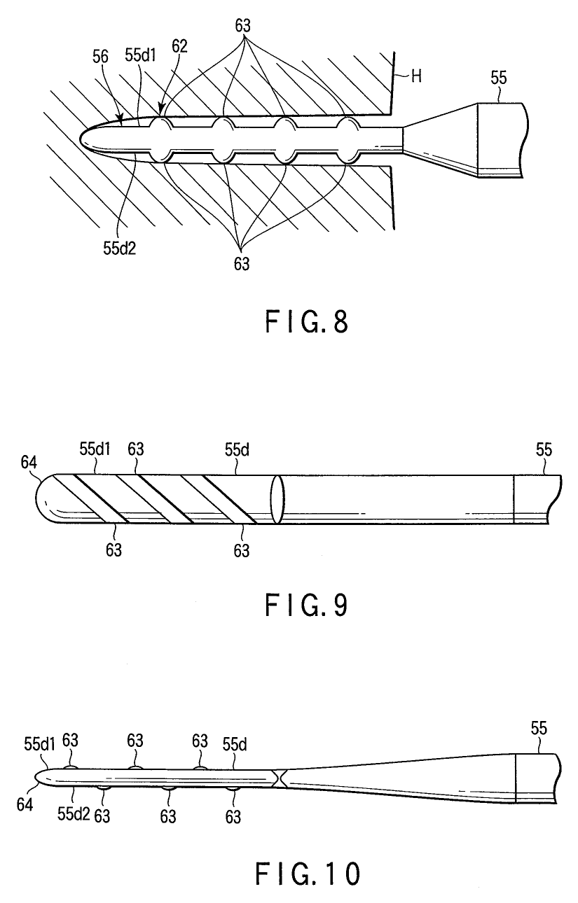

Next, the function of this embodiment configured as described above will be described below. When the surgical instrument 2 is to be used, the instrument 2 is set in advance in an assembled state where the operating part unit 22 is detachably and coaxially coupled to the handpiece 21 through the coupling mechanisms. In this state, by outputting a high-frequency current to the high-frequency cable 38 to supply the high-frequency current to the vibrator 24 and the probe 55, and press the operating part 56 of the probe 55 against the living tissue, it is possible to subject the living tissue to the high-frequency procedure. Further, by outputting a drive current to the piezoelectric element part 26 to vibrate the vibrator 24 and the probe 55 as one body by the ultrasonic vibration, and press the operating part 56 of the probe 55 against the living tissue, it is possible to subject the living tissue to the ultrasonic procedure. Accordingly, it is possible to simultaneously output the ultrasonic energy and the high-frequency energy from the blade 55d of the distal end of the probe 55. In this state, by inserting the blade 55d of the distal end of the probe 55 into the living tissue H as shown in FIG. 8, a procedure such as coagulation/incision or the like of a parenchymatous viscus (such as the liver or the like) is performed.

At the time of this procedure, when the blade 55d is inserted into the living tissue H, by bringing the plurality of convex parts 63 on the flat surfaces 55d1 and 55d2 on both sides of the blade 55d into contact with the wall surface of the living tissue H, it is possible to provide gaps between the flat surfaces 55d1 and 55d2 on both sides of the blade 55d and the wall surface of the living tissue H. As a result of this, it is possible to make the area of contact between the blade 55d and the living tissue H smaller. Thus, even when the living tissue H having a large area is brought into contact with the blade 55d of the distal end of the probe 55, it is possible to prevent the current density from being reduced, and hence it is possible to prevent the sharpness from being reduced by the diffusion of the current. As a result of this, the coagulating/incising capability of the surgical instrument 2 is not reduced.

Furthermore, at the time of a procedure such as coagulation/incision or the like of a parenchymatous viscus (such as the liver or the like), even when the living tissue H having a large area is brought into contact with the blade 55d of the distal end of the probe 55, it is possible to prevent the current density from being reduced, and hence it is not necessary to increase the set value of power. Thus, it is possible to prevent thermal invasion on the living tissue H from increasing.

Further, by applying ultrasonic vibration to the probe 55 in addition to the high-frequency current, it becomes hard for the living tissue H to stick to the probe. The living tissue does not stick to the probe, and hence it becomes possible to smoothly perform coagulation/incision without decreasing of the current density.

Further, when the ultrasonic vibration is transmitted to the probe 55, cavitation occurs at parts between the plurality of convex parts 63 provided on the probe 55. It is also possible to improve the incising capability of the surgical instrument 2 by utilizing the cavitation action. That is, when the living tissue H is subjected to an ultrasonic procedure, destruction of the tissue is promoted by the cavitation effect as indicated by arrows in FIG. 13. As a result of this, it is possible to support the sharpness of the surgical instrument 2. Thus, by virtue of the support of the cavitation for the sharpness, smoother coagulation/incision is enabled, and consequently, it is possible to suppress invasion on the living tissue H.

Thus, the surgical instrument configured as described above exerts the following effect. That is, as for the surgical instrument 2 of this embodiment, it is possible to provide a surgical instrument 2 which, when used in a state where the blade 55d of the distal end of the probe 55 is deeply inserted into the living tissue H, can maintain/improve the coagulating/incising capability without increasing the current and the voltage, and prevent the durability of the surgical instrument 2 from being deteriorated.

FIGS. 9 and 10 show a modification example of the probe 55 of the surgical instrument 2 of the first embodiment (see FIGS. 1 to 8). In the first embodiment, a configuration has been shown in which the plurality of convex parts 63 provided on the flat surfaces 55d1 and 55d2 on both sides of the blade 55d of the probe 55 are arranged side by side in the axial direction of the blade 55d in a state where the convex parts 63 are each extended in the direction perpendicular to the axial direction of the blade 55d. Conversely, in this modification example, as shown in FIG. 9, a plurality of convex parts 63 are arranged side by side in the axial direction of a blade 55d in a state where the convex parts 63 are each extended in an inclined direction inclined with respect to a direction perpendicular to the axial direction of the blade 55d.

Thus, in this modification example too, as in the first embodiment, when the blade 55d is inserted into the living tissue H, the plurality of convex parts 63 on the flat surfaces 55d1 and 55d2 on both sides of the blade 55d are brought into contact with a wall surface of the living tissue H, whereby it is possible to provide gaps between the flat surfaces 55d1 and 55d2 on both sides of the blade 55d and the wall surface of the living tissue H. This makes it possible to reduce the area of contact between the blade 55d and the living tissue H. Accordingly, even when the living tissue H having a large area is brought into contact with the blade 55d of the distal end of the probe 55, it is possible to prevent the current density from being reduced, and hence it is possible to prevent the sharpness from being reduced by the diffusion of the current. As a result of this, the coagulating/incising capability of the surgical instrument 2 is not reduced.

Furthermore, at the time of a procedure such as coagulation/incision or the like of a parenchymatous viscus (such as the liver or the like), even when the living tissue H having a large area is brought into contact with the blade 55d of the distal end of the probe 55, it is possible to prevent the current density from being reduced, and hence it is not necessary to increase the set value of power. Thus, it is possible to prevent thermal invasion on the living tissue H from increasing.

Further, when the ultrasonic vibration is transmitted to the probe 55, cavitation occurs at parts between the plurality of convex parts 63 provided on the probe 55. It is also possible to improve the incising capability of the surgical instrument 2 by utilizing the cavitation action.

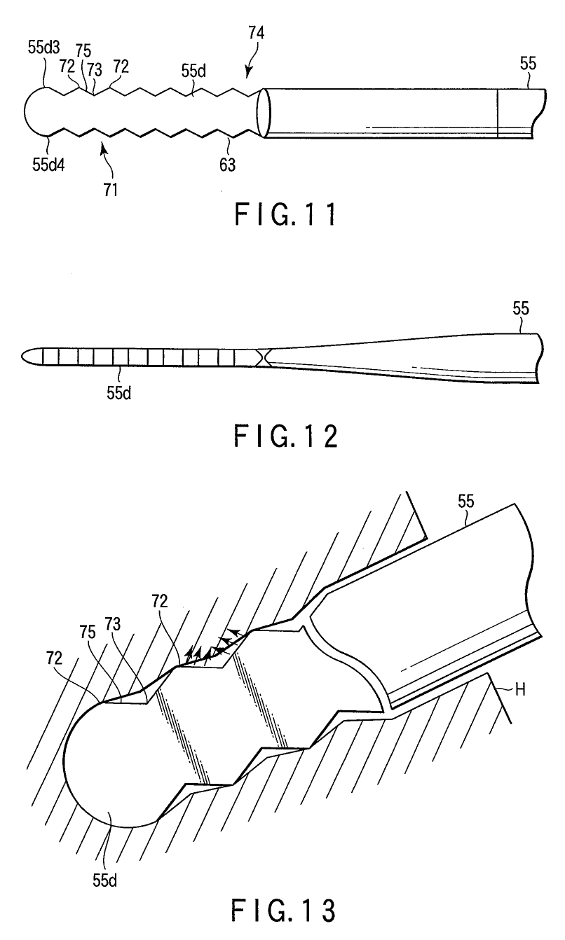

FIGS. 11 to 13 show a second embodiment of the present invention. This embodiment is formed by changing the configuration of the probe 55 of the surgical instrument 2 of the first embodiment (see FIGS. 1 to 8) as follows. The other configurations are identical with the first embodiment.

That is, in this embodiment, as shown in FIGS. 11 and 12, a probe 55 includes tooth parts 71 each having a sawtooth shape on side edge surfaces 55d3 and 55d4 on both sides of a blade 55d. The tooth parts 71 include a plurality of mountain-shaped convex parts 72 saliently provided on the side edge surfaces 55d3 and 55d4 on both sides of the blade 55d, and a plurality of valley-shaped concave parts 73 formed between adjacent convex parts 72. As a result of this, the plurality of convex parts 72 and the plurality of concave parts 73 are continuously and alternately arranged, whereby a contact area reduction part 74 for reducing the area of contact with the living tissue to thereby increase the current density of the high-frequency current is formed.

Between the convex part 72 and the concave part 73, an inclined surface 75 obliquely inclined with respect to the vibration direction (axial direction of the probe 55) of the ultrasonic vibration is provided. The vertex part of the convex part 72 is substantially made a point (line). The inclined surface 75 is formed into a shape having a width (area) from the vertex part of the convex part 72 toward the valley part of the concave part 73.

Thus, the surgical instrument configured as described above exerts the following effect. That is, in this embodiment, as shown in FIG. 13 the part of the probe to be in contact with the wall surface of the living tissue H is made each of the vertex parts of the sawtooth convex parts 72, and hence it is possible to provide gaps between the side edge surfaces 55d3 and 55d4 on both sides of the blade 55d and the wall surface of the living tissue H. This makes it possible to reduce the area of contact between the blade 55d and the living tissue H, and concentrate the current for the high-frequency procedure at the vertex parts of the sawtooth convex parts 72. Thus, even when the living tissue H having a large area is brought into contact with the blade 55d of the distal end of the probe 55, it is possible to prevent the current density from being reduced, and hence it is possible to prevent the current from being diffused. Accordingly, in the surgical instrument 2 of this embodiment, at the time of a procedure such as coagulation/incision or the like of a parenchymatous viscus (such as the liver or the like), it becomes possible to exert desired coagulating capability without increasing the power/voltage.

Furthermore, the tooth part 71 includes inclined surfaces 75 obliquely inclined with respect to the vibration direction (axial direction of the probe 55) of the ultrasonic vibration, and each of the inclined surfaces 75 is provided with a width (area). Thus, when the living tissue H is subjected to an ultrasonic procedure, destruction of the tissue is promoted by the cavitation effect as indicated by arrows in FIG. 13. As a result of this, it is possible to support the sharpness of the surgical instrument 2. Thus, by virtue of the support of the cavitation for the sharpness, smoother coagulation/incision is enabled, and consequently, it is possible to suppress invasion on the living tissue H.

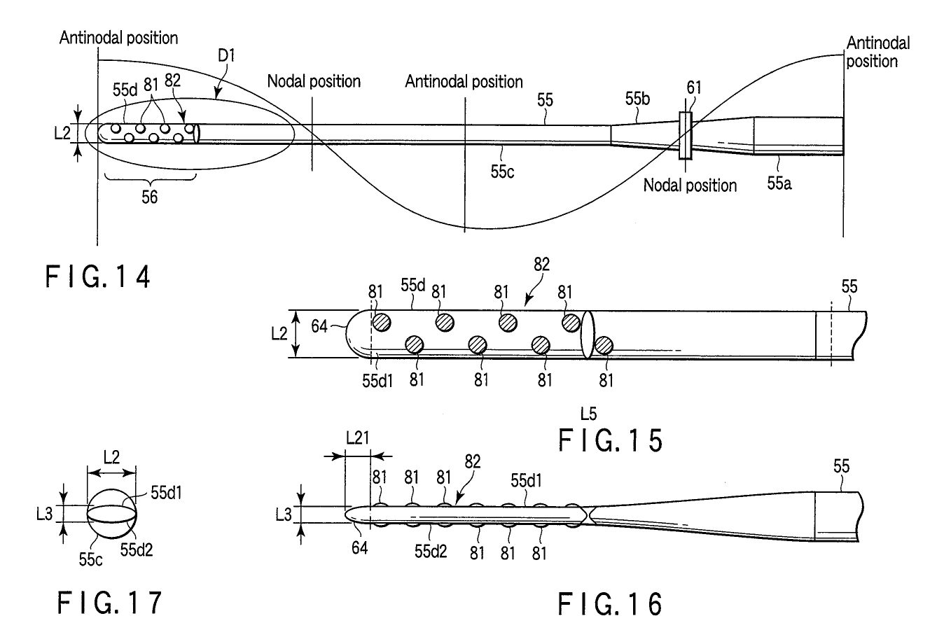

FIGS. 14 to 17 show a third embodiment of the present invention. This embodiment is formed by changing the configuration of the probe 55 of the surgical instrument 2 of the first embodiment (see FIGS. 1 to 8) as follows. The other configurations are identical with the first embodiment.

That is, in the probe 55 of this embodiment, as shown in FIGS. 14 to 16, a plurality of hemispherical protruding parts 81 are saliently provided on flat surfaces 55d1 and 55d2 on both sides of a blade 55d. On one flat surface 55d1 side, as shown in FIG. 15, a plurality of (in this embodiment, four in one row) protruding parts 81 are provided in two rows arranged above and below with respect to the center line of the probe 55 in FIG. 15. Furthermore, the protruding parts 81 on the upper row, and the protruding parts 81 on the lower row are arranged in a state where the two rows are shifted from each other in the longitudinal direction, whereby the protruding parts 81 are arranged in a staggered layout as a whole. On the other flat surface 55d2 side too, a plurality of protruding parts 81 are arranged in the same way. These protruding parts 81 constitute a contact area reduction part 82 for reducing the area of contact with the living tissue to thereby increase the current density of the high-frequency current.

Furthermore, in this embodiment, a length (L21) between a distal end of the blade 55d and the protruding part 81 at the forefront position is 1.5 mm ((L21)=1.5 mm). Further, the protruding parts 81 are arranged in the vicinity of the antinodal position of the ultrasonic vibration, e.g., within a range of a quarter-wave length of the ultrasonic vibration from the distal end position of the blade 55d.

Thus, the surgical instrument configured as described above exerts the following effect. That is, in this embodiment, the part of the probe to be in contact with the wall surface of the living tissue H is made each of the vertex parts of the protruding parts 81 of the contact area reduction part 82, and hence it is possible to provide gaps between both the side surfaces of the blade 55d and the wall surface of the living tissue H. This makes it possible to reduce the area of contact between the blade 55d and the living tissue H, and concentrate the current for the high-frequency procedure at the vertex parts of the protruding parts 81. Thus, even when the living tissue H having a large area is brought into contact with the blade 55d of the distal end of the probe 55, it is possible to prevent the current density from being reduced, and hence it is possible to prevent the current from being diffused. Accordingly, in the surgical instrument 2 of this embodiment, at the time of a procedure such as coagulation/incision or the like of a parenchymatous viscus (such as the liver or the like), it becomes possible to exert desired coagulating capability without increasing the power/voltage.

Furthermore, the hemispherical protruding parts 81 includes spherical surfaces 75 obliquely inclined with respect to the vibration direction (axial direction of the probe 55) of the ultrasonic vibration. Thus, when the living tissue H is subjected to an ultrasonic procedure, destruction of the tissue is promoted by the cavitation effect. As a result of this, it is possible to support the sharpness of the surgical instrument 2. Thus, by virtue of the support of the cavitation for the sharpness, smoother coagulation/incision is enabled, and consequently, it is possible to suppress invasion on the living tissue H.

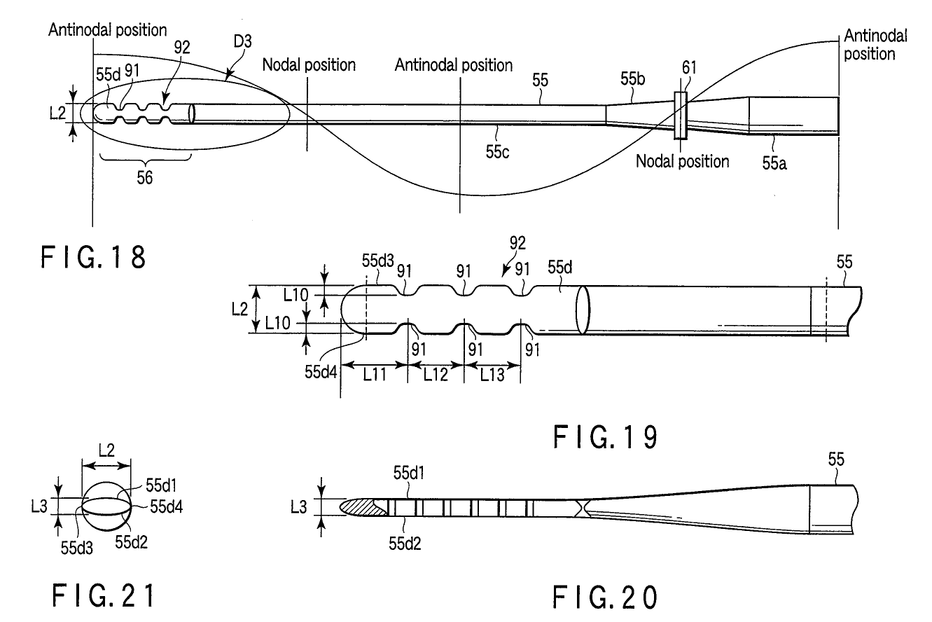

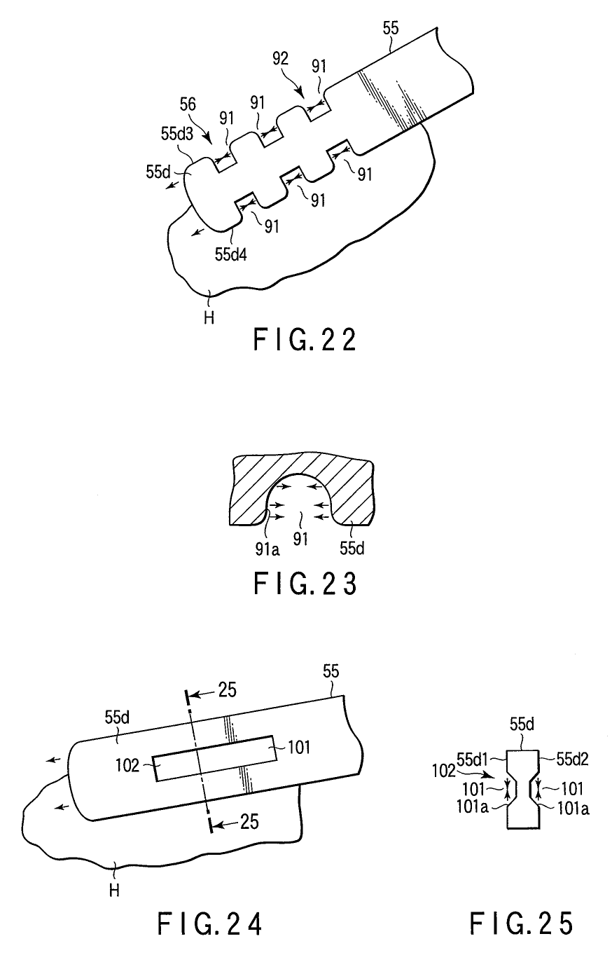

FIGS. 18 to 23 show a fourth embodiment of the present invention. This embodiment is formed by changing the configuration of the probe 55 of the surgical instrument 2 of the first embodiment (see FIGS. 1 to 8) as follows. The other configurations are identical with the first embodiment.

That is, in this embodiment, as shown in FIGS. 18 and 19, a probe 55 includes a plurality of (three in this embodiment) concave parts 91 each of which is inwardly depressed on side edge surfaces 55d3 and 55d4 on both sides of a blade 55d. The concave parts 91 are arranged on both the side edge surfaces 55d3 and 55d4 of the blade 55d with axial direction of the probe 55. Furthermore, it is desirable that as shown in FIG. 23, the part of the concave part 91 of the blade 55d be provided with an inclined surface 91a obliquely inclined with respect to the vibration direction (axial direction of the probe 55) of the ultrasonic vibration.

Further, in this embodiment, the concave parts 91 on both the side edge surfaces 55d3 and 55d4 of the blade 55d are arranged at positions of bilateral symmetry. This enables prevention of transverse vibration of ultrasonic vibration.

In this embodiment, when the blade 55d is brought into contact with the wall surface of the living tissue H, it is possible to produce parts at which the blade 55d is not brought into contact with the wall surface of the living tissue H on both the side edge surfaces 55d3 and 55d4 of the blade 55d by the parts of the concave parts 91 of the blade 55d. As a result of this, a contact area reduction part 92 for reducing the area of contact with the living tissue to thereby increase the current density of the high-frequency current is formed.

Furthermore, in this embodiment, a depth (L10) of the concave part 91 is 0.5 mm ((L10)=0.5 mm). A length (L11) between a distal end of the blade 55d and a center position of the concave part 91 at the forefront position is 4 mm ((L11)=4 mm). A length (L12) between the center position of the concave part 91 at the forefront position and a center position of the second concave part 91 from the distal end side is 3.5 mm ((L12)=3.5 mm). A length (L13) between the center position of the second concave part 91 from the distal end side and a center position of the third concave part 91 from the distal end side is 3.5 mm ((L13)=3.5 mm). Further, the concave parts 91 are arranged in the vicinity of the antinodal position of the ultrasonic vibration, e.g., within a range of a quarter-wave length of the ultrasonic vibration from the distal end position of the blade 55d.

Further, a value 2×(L10)/(L3) is set at 0.2 to 0.4 (2×(L10)/(L3)=0.2 to 0.4). This makes it possible for the blade 55d to secure the strength margin in performing the ultrasonic vibration.

Thus, the surgical instrument configured as described above exerts the following effect. That is, in this embodiment, when the blade 55d is brought into contact with the wall surface of the living tissue H, it is possible to produce parts at which the blade 55d is not brought into contact with the wall surface of the living tissue H on both the side edge surfaces 55d3 and 55d4 of the blade 55d by the parts of the concave parts 91 of the blade 55d. Thus, it is possible to make the area of contact between both the side edge surfaces 55d3 and 55d4 of the blade 55d and the wall surface of the living tissue H small. This makes it possible to concentrate the current for the high-frequency procedure at the parts of contact between both the side edge surfaces 55d3 and 55d4 of the blade 55d and the wall surface of the living tissue H. Thus, even when the living tissue H having a large area is brought into contact with the blade 55d of the distal end of the probe 55, it is possible to prevent the current density from being reduced, and hence it is possible to prevent the current from being diffused. Accordingly, in the surgical instrument 2 of this embodiment, at the time of a procedure such as coagulation/incision or the like of a parenchymatous viscus (such as the liver or the like), it becomes possible to exert desired coagulating capability without increasing the power/voltage.

Furthermore, when an inclined surface 91a obliquely inclined with respect to the vibration direction (axial direction of the probe 55) of the ultrasonic vibration is provided at a part of the concave part 91 of the blade 55d, at the time of subjecting the living tissue H to an ultrasonic procedure, it is possible to produce cavitation by the part of the inclined surface 91a of the concave part 91 of the blade 55d as indicated by the arrows in FIG. 23. Thus, destruction of the tissue is promoted by the cavitation effect, and hence it is possible to support the sharpness of the surgical instrument 2. As a result of this, by virtue of the support of the cavitation for the sharpness, smoother coagulation/incision is enabled, and consequently, it is possible to suppress invasion on the living tissue H.

FIGS. 24 and 25 each show a modification example of the probe 55 of the surgical instrument 2 of the fourth embodiment (see FIGS. 18 to 23). A probe 55 of this modification example is provided with inwardly depressed concave parts 101 on flat surfaces 55d1 and 55d2 on both sides of a blade 55d as shown in FIG. 25. As shown in FIG. 24, the concave part 101 is arranged on the center line of the probe 55, and is formed into an elongated hole (groove) elongated in the axial direction of the probe 55. Furthermore, it is desirable that the part of the concave part 101 of the blade 55d be provided with an inclined surface 101a inclined with respect to the vibration direction (axial direction of the probe 55) of the ultrasonic vibration.

In this modification example, it is possible, when the blade 55d is brought into contact with the wall surface of the living tissue H, to provide parts which are not brought into contact with the wall surface of the living tissue H on both the side surfaces 55d1 and 55d2 of the blade 55d by the parts of the concave parts 101 of the blade 55d. As a result of this, a contact area reduction part 102 for reducing the area of contact with the living tissue to thereby increase the current density of the high-frequency current is formed.

Thus, in the probe 55 of this modification example, it is possible, when the blade 55d is brought into contact with the wall surface of the living tissue H, to provide parts which are not brought into contact with the wall surface of the living tissue H on both the side surfaces 55d1 and 55d2 of the blade 55d by the parts of the concave parts 101 of the blade 55d. As a result of this, it is made possible to make the area of contact between both the side surfaces 55d1 and 55d2 of the blade 55d and the wall surface of the living tissue H small. This makes it possible to concentrate the current for the high-frequency procedure at the parts of contact between both the side surfaces 55d1 and 55d2 of the blade 55d and the wall surface of the living tissue H. Thus, even when the living tissue H having a large area is brought into contact with the blade 55d of the distal end of the probe 55, it is possible to prevent the current density from being reduced, and hence it is possible to prevent the current from being diffused. Accordingly, in the surgical instrument 2 of this modification example, at the time of a procedure such as coagulation/incision or the like of a parenchymatous viscus (such as the liver or the like), it becomes possible to exert desired coagulating capability without increasing the power/voltage.

Furthermore, when the part of each of the concave parts 101 of the blade 55d is provided with inclined surfaces 101a obliquely inclined with respect to the vibration direction (axial direction of the probe 55) of the ultrasonic vibration, it is possible, at the time of subjecting the living tissue H to an ultrasonic procedure, to produce cavitation by the parts of the inclined surfaces 101a of each of the concave parts 101 of the blade 55d as indicated by the arrows in FIG. 25. Thus, destruction of the tissue is promoted by the cavitation effect, and hence it is possible to support the sharpness of the surgical instrument 2. As a result of this, by virtue of the support of the cavitation for the sharpness, smoother coagulation/incision is enabled, and consequently, it is possible to suppress invasion on the living tissue H.

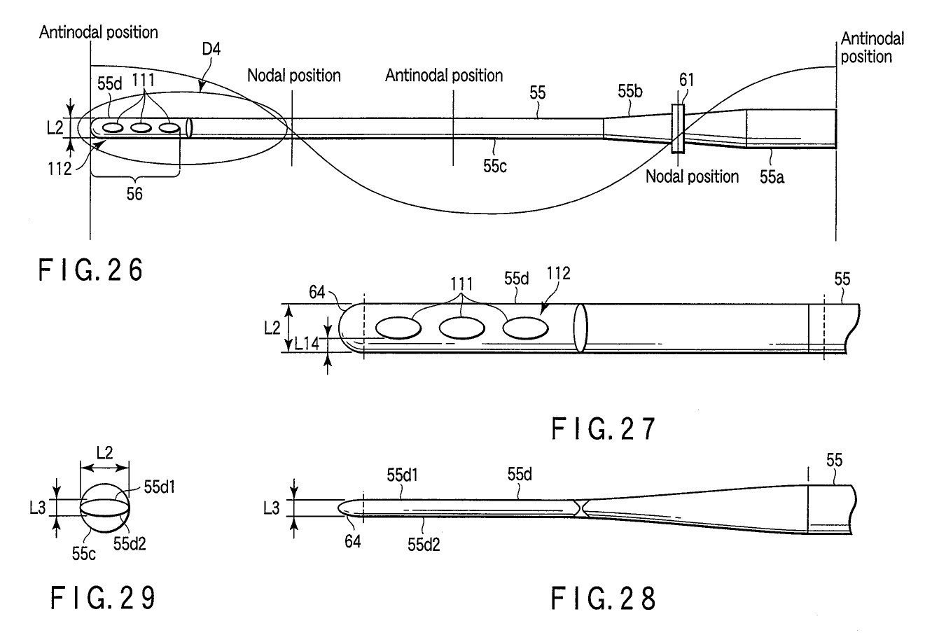

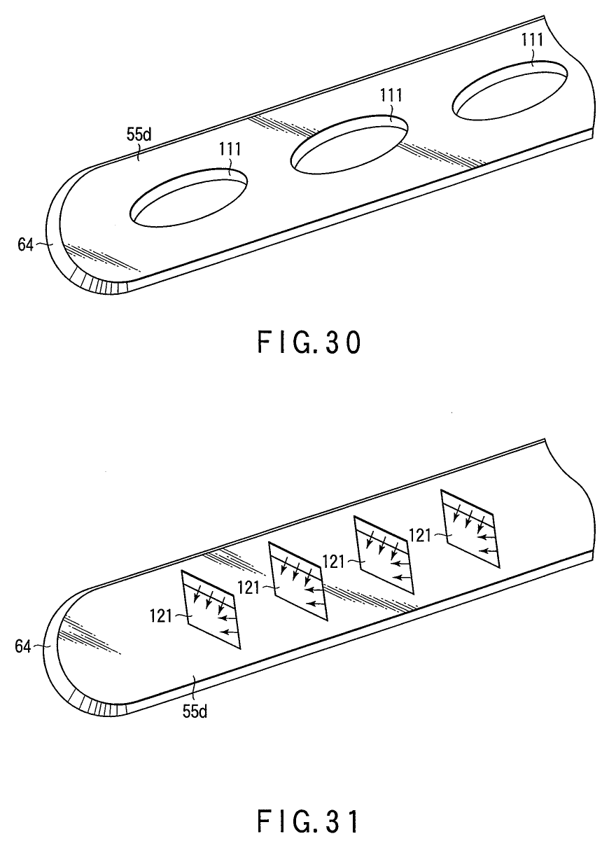

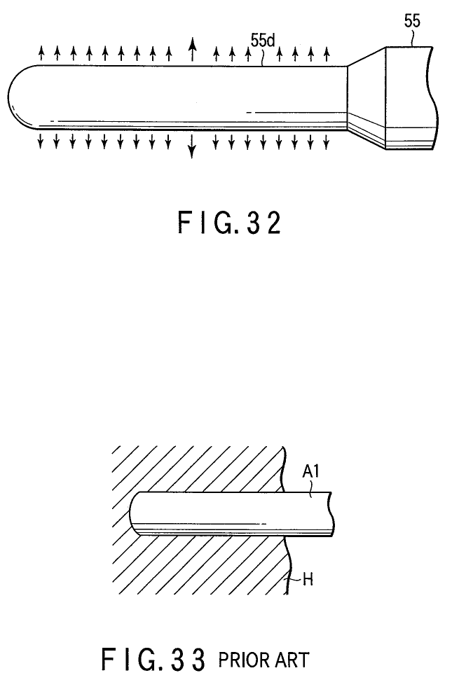

FIGS. 26 to 30 show a fifth embodiment of the present invention. This embodiment is formed by changing the configuration of the probe 55 of the surgical instrument 2 of the first embodiment (see FIGS. 1 to 8) as follows. The other configurations are identical with the first embodiment.

That is, a probe 55 of this embodiment includes, as shown FIGS. 26 and 27, a plurality of (three in this embodiment) hole parts 111 penetrating a blade 55d from one of flat surfaces 55d1 and 55d2 on both sides of the blade 55d to the other. The hole parts 111 are arranged in line along the center axis of the blade 55d. Each of the hole parts 111 is formed into an elliptical shape elongated in the center axis direction of the blade 55d. It should be noted that the hole part 111 may be shaped oval.

In this embodiment, it is possible, when the blade 55d is brought into contact with the wall surface of the living tissue H, to provide parts which are not brought into contact with the wall surface of the living tissue H on both the side surfaces 55d1 and 55d2 of the blade 55d by the parts of the hole parts 111 of the blade 55d. As a result of this, a contact area reduction part 112 for reducing the area of contact with the living tissue to thereby increase the current density of the high-frequency current is formed.

Furthermore, in this embodiment, a width (L14) between both the side surfaces 55d1 and 55d2, and the hole part 111 is set at 1 mm ((L14)=1 mm). Further, the hole parts 111 are arranged in the vicinity of the antinodal position of the ultrasonic vibration, e.g., within a range of a quarter-wave length of the ultrasonic vibration from the distal end position of the blade 55d.

Thus, the surgical instrument configured as described above exerts the following effect. That is, in the probe 55 of this embodiment, when the blade 55d is brought into contact with the wall surface of the living tissue H, it is possible to produce parts at which the blade 55d is not brought into contact with the wall surface of the living tissue H on both the side surfaces 55d1 and 55d2 of the blade 55d by the parts of the hole parts 111 of the blade 55d. Thus, it is possible to make the area of contact between both the side surfaces 55d1 and 55d2 of the blade 55d and the wall surface of the living tissue H small. This makes it possible to concentrate the current for the high-frequency procedure at the parts of contact between both the side surfaces 55d1 and 55d2 of the blade 55d and the wall surface of the living tissue H. Thus, even when the living tissue H having a large area is brought into contact with the blade 55d of the distal end of the probe 55, it is possible to prevent the current density from being reduced, and hence it is possible to prevent the current from being diffused. Accordingly, in the surgical instrument 2 of this embodiment, at the time of a procedure such as coagulation/incision or the like of a parenchymatous viscus (such as the liver or the like), it becomes possible to exert desired coagulating capability without increasing the power/voltage.

FIG. 31 shows a modification example of the insertion part of the surgical instrument 2 of the fifth embodiment (see FIGS. 26 to 30). In this modification example, four through-hole parts 121 each having a rhombic shape are formed between flat surfaces 55d1 and 55d2 on both sides of a blade 55d. The hole parts 121 are provided in line along the center axis of the blade 55d. The hole part 121 has a rhombic shape a major axis of which is arranged in a direction perpendicular to the center axis direction of the blade 55d.

Thus, the surgical instrument configured as described above exerts the following effect. That is, in the probe 55 of this modification example, when the blade 55d is brought into contact with the wall surface of the living tissue H, it is possible to produce parts at which the blade 55d is not brought into contact with the wall surface of the living tissue H on both the side surfaces 55d1 and 55d2 of the blade 55d by the parts of the hole parts 121 of the blade 55d. Thus, it is possible to make the area of contact between both the side surfaces 55d1 and 55d2 of the blade 55d and the wall surface of the living tissue H small. This makes it possible to concentrate the current for the high-frequency procedure at the parts of contact between both the side surfaces 55d1 and 55d2 of the blade 55d and the wall surface of the living tissue H. Thus, even when the living tissue H having a large area is brought into contact with the blade 55d of the distal end of the probe 55, it is possible to prevent the current density from being reduced, and hence it is possible to prevent the current from being diffused. Accordingly, in the surgical instrument 2 of this modification example, at the time of a procedure such as coagulation/incision or the like of a parenchymatous viscus (such as the liver or the like), it becomes possible to exert desired coagulating capability without increasing the power/voltage.

Furthermore, in this modification example, the hole part 121 of the blade 55d has a rhombic hole shape, and hence it is a hole shape having a large area perpendicular to the vibration direction of the ultrasonic vibration. Accordingly, cavitation is easily produced, and thus an effect of enabling a procedure attaching importance to incising capability is obtained by utilizing the cavitation action.

FIG. 32 shows a sixth embodiment of the present invention. This embodiment is formed by changing the configuration of the probe 55 of the surgical instrument 2 of the first embodiment (see FIGS. 1 to 8) as follows. The other configurations are identical with the first embodiment.

That is, this embodiment is configured in such a manner that ultrasonic vibration in a transverse vibration mode is transmitted to a blade 55d of a distal end of a probe 55 of a surgical instrument 2 as indicated by arrows in FIG. 32.

In this embodiment, cavitation is easily produced in the incision direction (direction perpendicular to the axial direction of the blade 55d), and hence an effect of enabling a procedure attaching importance to incising capability is obtained by utilizing the cavitation action.

It should be noted that the present invention is not limited to the embodiments described above, and it goes without saying that the invention can be variously modified and implemented within the scope not deviating from the gist thereof.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

1. A surgical operation apparatus comprising:
a probe to which ultrasonic vibration is transmitted; and
a flat plate-like blade which is formed at a distal end part of the probe, and can simultaneously output ultrasonic vibration and a high-frequency current, wherein
the blade includes an area for reducing an area of contact with the living tissue to thereby increase a current density of the high-frequency current.

2. The surgical operation apparatus according to claim 1, wherein
the area includes, on flat surfaces on both sides of the blade, convex parts outwardly protruded from positions on each of the flat surfaces, and
each of the convex parts is provided to extend in at least one of a direction perpendicular to an axial direction of the blade, and a direction obliquely intersecting the axial direction of the blade.

3. The surgical operation apparatus according to claim 2, wherein
as the convex parts, a plurality of linear protruding parts extended in a direction perpendicular to the axial direction of the blade are arranged in line in the axial direction of the blade on the flat surfaces on both sides of the blade.

4. The surgical operation apparatus according to claim 2, wherein
as the convex parts, a plurality of linear protruding parts extended in an oblique direction obliquely inclined with respect to a direction perpendicular to the axial direction of the blade are arranged in line in the axial direction of the blade on the flat surfaces on both sides of the blade.

5. The surgical operation apparatus according to claim 2, wherein
as the convex parts, a plurality of hemispherical protruding parts are saliently provided on the flat surfaces on both sides of the blade.

6. The surgical operation apparatus according to claim 1, wherein
the area includes inwardly depressed concave parts on the flat surfaces on both sides of the blade.

7. The surgical operation apparatus according to claim 6, wherein
the concave part includes a flat surface in at least one of a direction perpendicular to the axial direction of the blade, and a direction obliquely intersecting the axial direction of the blade.

8. The surgical operation apparatus according to claim 6, wherein
the concave part is able to produce cavitation when the ultrasonic vibration is output.

9. The surgical operation apparatus according to claim 1, wherein
the area includes tooth parts each of which has a sawtooth shape and which are formed by continuously providing a plurality of convex parts and concave parts alternately on the outside of both edge surfaces of the blade.

10. The surgical operation apparatus according to claim 1, wherein
the area includes a hole part penetrating the blade from one of flat surfaces on both sides of the blade to the other.

11. The surgical operation apparatus according to claim 10, wherein
the hole part is arranged on a center axis of the blade.

12. The surgical operation apparatus according to claim 11, wherein
a plurality of the hole parts are provided along the center axis of the blade.

13. The surgical operation apparatus according to claim 12, wherein
the hole part has an oval shape or an elliptical shape elongated in the center axis direction of the blade.

14. The surgical operation apparatus according to claim 12, wherein
the hole part has a rhombic shape a major axis of which is arranged in a direction perpendicular to the center axis direction of the blade.

15. The surgical operation apparatus according to claim 1, wherein
in the probe, an antinodal position of the ultrasonic vibration is set at a distal end of the blade, and
the area is formed within a range of a quarter-wave length of the ultrasonic vibration from the distal end position of the blade.
